(19) United States
(12) Patent Application Publication
(10) Pub. No.: US 2015/0296953 A1
(43) Pub. Date: Oct. 22, 2015

(54) HAIR TREATMENT APPLICATION DELIVERY SYSTEM COMPRISING A COMPOSITION

(71) Applicant: The Procter & Gamble Company, Cincinnati, OH (US)

(72) Inventors: William Dale MURDOCK, Cheviot, OH (US); Mikah COFFINDAFFER, Mason, OH (US); James Douglas STILL, Cleves, OH (US); David Arthur STURGIS, Cincinnati, OH (US); Kendrick Jon HUGHES, Cincinnati, OH (US); Mannie Lee CLAPP, Cincinnati, OH (US); Christopher IRWIN, Cincinnati, OH (US)

(21) Appl. No.: 14/693,357

(22) Filed: Apr. 22, 2015

Related U.S. Application Data
(60) Provisional application No. 61/982,600, filed on Apr. 22, 2014.

(51) Int. Cl. A45D 19/16; A45D 24/22; A61Q 5/06; A45D 40/26; A61K 8/92; A61K 8/25

(52) U.S. Cl. CPC A45D 19/16; A61K 8/92; A61K 8/25; A61Q 5/06; A45D 40/26; A45D 24/22; A61K 2800/30

(57) ABSTRACT

A hair treatment application delivery system directed to at least one solid treatment composition comprising a wax and a silicone; and a hair treatment application delivery device comprising a first plate and a second plate positionable in a juxtaposed relationship when the hair treatment application delivery device is in a closed state, wherein the first and second plates are coupled together via a connection, and wherein the first and second plates each has an internal and external surface.

### FIELD OF THE INVENTION

The present invention relates to a hair treatment application delivery system which comprises a treatment composition and an application device which is compact and portable and allows for easy, precise and non-messy targeted application to the hair.

### BACKGROUND OF THE INVENTION

Anti-frizz/flyaway reduction is a large benefit focus area for leave on treatment users as a recent study indicated that 64% of these consumers in the US are concerned about fly-aways throughout the day, wherein 62% where interested in using a product for flyaways throughout the day. Currently, to treat and apply anti-frizz/split-end treatments consumers must have bottle and use their hands/fingers to apply the product to the hair. The current frizz/split-end products generally have to be applied to wet hair before styling and are not intuitively targeted, leaving the roots too greasy and the ends too dry. This process for treating frizz/flyaways is messy, leaving excess product and natural shedding of hairs on the consumers' hands requiring them to also wash their hands after application. The packaging for this type product is not designed for portability or compactness and has been known to leak/completely open when transported in a bag/purse. There are currently no product/delivery systems designed for targeted application and for use throughout the day on dry hair when consumers really notice frizz or split ends.

The present invention will provide the consumer with a means to treat their hair frizz/flyaways conveniently and easily. The invention consists of a compact, portable device containing a solid formulation that will provide targeted delivery of a solution for hair frizz/flyaways/smoothing on dry hair throughout the day. The design is such that the consumer can have the portable filled device with them all day, use whenever needed to treat their dry hair, and due to the device and solid formula, experience no mess on their hands or fingers while achieving hair benefit previously only possible on wet hair.

These, and other objects, will become readily apparent from the detailed description below.

### SUMMARY OF THE INVENTION

In an embodiment of the present invention, it is directed to a hair treatment application delivery system comprising at least one solid treatment composition comprising a wax and a silicone; and a hair treatment application delivery device comprising a first plate and a second plate positionable in a juxtaposed relationship when the hair treatment application delivery device is in a closed state, wherein the first and second plates are coupled together via a connection, and wherein the first and second plates each has an internal and external surface.

### BRIEF DESCRIPTION OF THE DRAWINGS

FIG. 1 is a perspective view of one embodiment of a hair treatment application delivery system of the present invention. The hair treatment application delivery system comprises a first (2) and a second (3) plate coupled together a connection (8). The second plate (3) comprises an internal surface (5). The first plate (2) comprises an external surface (6).

FIG. 2 is a front view of one embodiment of a hair treatment application delivery system of the present invention. The hair treatment application delivery system comprises an internal surface (4) of a first plate (2). The second plate (3) comprises an external surface (7). The first plate (2) and the second plate (3) comprise a substrate (10). The first plate (2) comprises a depression(s) (11).

FIG. 3 is a side view of one embodiment of a hair treatment application delivery system of the present invention and a hair treatment treatment application device (1).

FIG. 4 is a back view of one embodiment of a hair treatment application delivery system of the present invention. The hair treatment application delivery system comprises a locking mechanism (9).

FIG. 5 is a perspective view of one embodiment of a hair treatment application delivery system of the present invention. The hair treatment delivery system comprises a hinge spring (12) and a sliding lock feature (13).

FIG. 6 is a perspective view of one embodiment of a hair treatment application delivery system of the present invention. The hair treatment delivery system comprises a first plate (2) and a second plate (3).

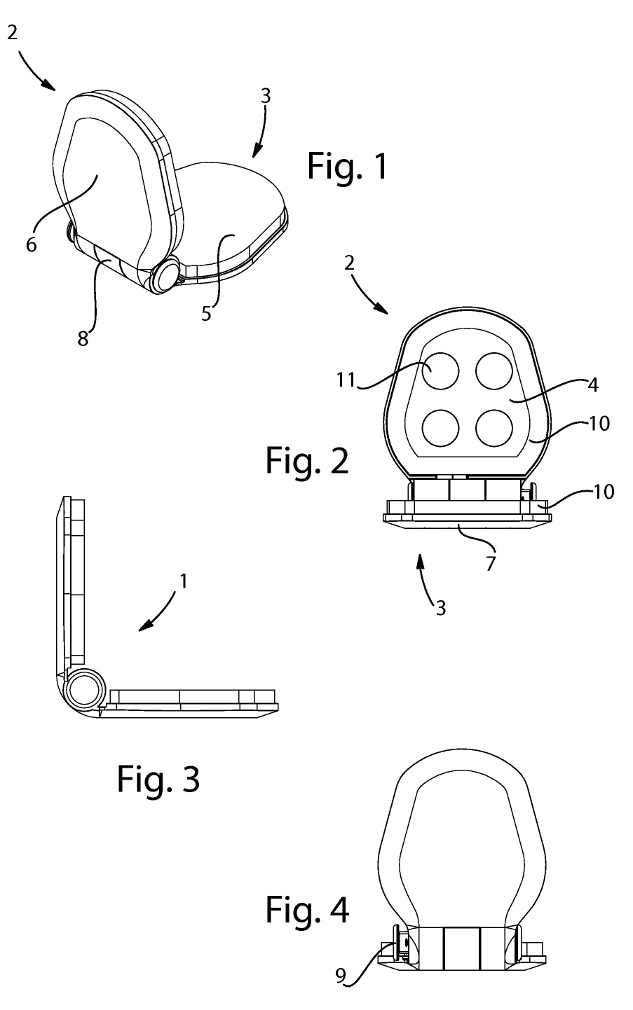

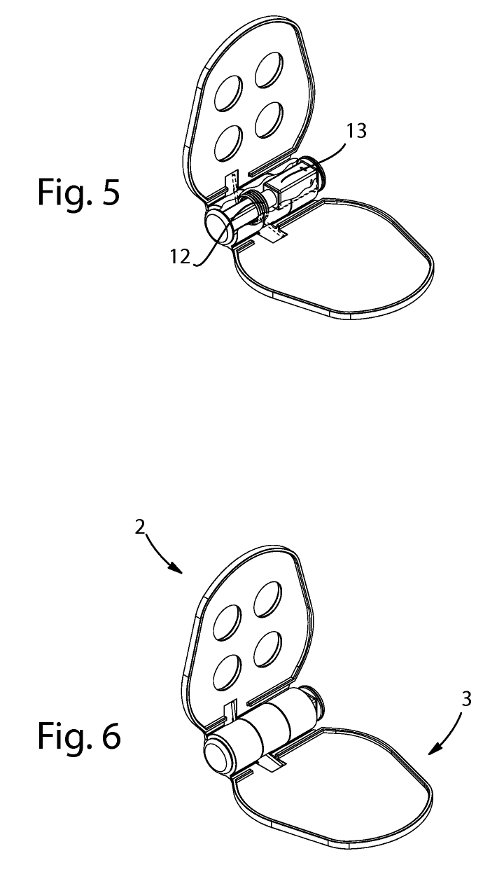

### DETAILED DESCRIPTION OF EMBODIMENTS OF THE INVENTION

All percentages and ratios used herein are by weight of the total composition, unless otherwise designated. All measurements are understood to be made at ambient conditions, where “ambient conditions” means conditions at about 25° C., under about one atmosphere of pressure, and at about 50% relative humidity, unless otherwise designated. All numeric ranges are inclusive of narrower ranges; delineated upper and lower range limits are combinable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

“Apply” or “application,” as used in reference to a composition, means to apply or spread the compositions of the present invention onto keratinous tissue such as the hair.

“Dermatologically acceptable” means that the compositions or components described are suitable for use in contact with human skin tissue without undue toxicity, incompatibility, instability, allergic response, and the like.

“Safe and effective amount” means an amount of a compound or composition sufficient to significantly induce a positive benefit.

“Leave-on,” in reference to compositions, means compositions intended to be applied to and allowed to remain on the keratinous tissue. These leave-on compositions are to be distinguished from compositions, which are applied to the hair and subsequently (in a few minutes or less) removed either by washing, rinsing, wiping, or the like. Leave-on compositions exclude rinse-off applications such as shampoos, rinse-off conditioners, facial cleansers, hand cleansers, body wash, or body cleansers. The leave-on compositions may be substantially free of cleansing or detersive surfactants. For example, “leave-on compositions” may be left on the keratinous tissue for at least 15 minutes. For example, leave-on compositions may comprise less than 1% detersive surfactants, less than 0.5% detersive surfactants, or 0% detersive surfactants. The compositions may, however, contain emulsifying, dispersing or other processing surfactants that are not intended to provide any significant cleansing benefits when applied topically to the hair.

“Soluble” means at least about 0.1 g of solute dissolves in 100 ml of solvent, at 25° C. and 1 atm of pressure

All percentages are by weight of the total composition, unless stated otherwise. All ratios are weight ratios, unless specifically stated otherwise. All ranges are inclusive and combinable. The number of significant digits conveys neither a limitation on the indicated amounts nor on the accuracy of the measurements. The term “molecular weight” or “M.Wt.” as used herein refers to the weight average molecular weight unless otherwise stated. The weight average molecular weight may be measured by gel permeation chromatography “QS” means sufficient quantity for 100%.

The term “substantially free from” or “substantially free of” as used herein means less than about 1%, or less than about 0.8%, or less than about 0.5%, or less than about 0.3%, or about 0%, by total weight of the composition.

“Hair,” as used herein, means mammalian hair including scalp hair, facial hair and body hair, particularly on hair on the human head and scalp.

“Cosmetically acceptable,” as used herein, means that the compositions, formulations or components described are suitable for use in contact with human keratinous tissue without undue toxicity, incompatibility, instability, allergic response, and the like. All compositions described herein which have the purpose of being directly applied to keratinous tissue are limited to those being cosmetically acceptable.

“Derivatives,” as used herein, includes but is not limited to, amide, ether, ester, amino, carboxyl, acetyl, acid, salt and/or alcohol derivatives of a given compound.

“Polymer,” as used herein, means a chemical formed from the polymerisation of two or more monomers. The term “polymer” as used herein shall include all materials made by the polymerisation of monomers as well as natural polymers. Polymers made from only one type of monomer are called homopolymers. A polymer comprises at least two monomers. Polymers made from two or more different types of monomers are called copolymers. The distribution of the different monomers can be calculated statistically or block-wise—both possibilities are suitable for the present invention. Except if stated otherwise, the term “polymer” used herein includes any type of polymer including homopolymers and copolymers.

1. Hair Treatment Application Device

1. Plates

In an embodiment of the present invention, the hair treatment application device (1) has a first (2) and a second plate (3); both plates (2;3) are of ergonomic size and can thus fit easily on either hand. The shape of the plates may vary. Rectangular, square, circular, elliptical, oval or oblong shapes may be useful as they are easy to manufacture but other shapes, particularly those that are easily recognised by the consumers may be used. As shown in Fig. X, each plate (2;3) comprises an internal (4;5) and external surface (6;7).

The internal (4;5) and/or external (6;7) surfaces of each plate (2;3) may be independently flat or curved. In an embodiment of the present invention, each plate or at least a portion thereof may comprise or one or more grooves. Said grooves may be independently located on either or both the internal and external surfaces (4;5;6;7) of either or both plates (2;3).

Each plate may be of the same or different size and shape. In an embodiment of the present invention, the shape of the first plate is the mirror image of the shape of the second plate (2;3) for easy manufacture.

Each internal surface (4;5) of said plates (2;3) may have an area of from about 2 cm² to about 70 cm², in an embodiment of the present invention from about 3 cm² to about 50 cm², in a further embodiment from about 4 cm² to about 30 cm².

In a further embodiment of the present invention, a first plate (2) may be present with no second plate (3). In a further embodiment, such first plate (2) may contain both a hair treatment composition and a substrate (10). Further, a first plate (2) and a second plate (3) may be present with no connection (8).

In certain embodiments, at least a portion of said first plate (2) and/or second plate (3) may comprise a depression formed in said first plate (2), preferably a concave depression. In certain other embodiments, at least a portion of said first (2) and/or second plate (3) may form a cavity, preferably a concave cavity. Combination of cavities, grooves and depression may also be present within the same hair treatment application device (1). In an embodiment of the present invention, at least a portion of said first plate (2) and/or second plate (3) may comprise a cavity which will hold the hair treatment composition. In a further embodiment, at least a portion of said first plate (2) and/or second plate (3) may comprise an area for affixing a hair treatment composition.

In an embodiment of the present invention, the internal surface of the first plate and/or second plate may comprise a depression(s) (11), to aid in anchoring the hair treatment composition to the device. In an embodiment, such anchoring features can further be raised or depressed and may include words, symbols, or tines.

In an embodiment, the hair treatment composition may be affixed to a first or second plate by means such as and including two-side tape, thermo-set, hot melt and cold seal, adhesion or extrusion lamination. Mechanical interlock or entanglement such as Velcro®, clamping, snap locks, sealing beads, locking pins and magnetism may also be used to adhere the hair treatment composition to the a first or second plate. Further, in order to maintain the device in a closed position, the device may contain a mechanical interlock or entanglement such as Velcro®, clamping, snap locks, sealing beads, locking pins and magnetism.

The external surface (6;7) of said first and/or second plate (2;3) may further comprise one or more areas, which have visible or tactile differences from said internal surfaces (4;5). Said visible or tactile differences comprise differences in colour or shade, differences in patterns, markings or embossments. The molded features can be a combination of thermoplastic elastomer (TPE)/soft touch materials, embossing and/or de-bossing of the surfaces, optimized to provide a reliable grip area on each half of the device. Such soft material may include soft injection moldable resins; Thermoplastic elestomers; TPE including styrenic (SEBS and SBS) based and olefin (TPO PP Elastomer) based; Thermoplastic eurothanes TPU; Melt Processable Rubber MPR; Thermoplastic Vulcanizate TPV; and Poly vinyl chloride PVC and mixtures thereof. In an embodiment of the present invention, the soft like material may delight the consumer with a soft touch feel while opening and closing the hair treatment application delivery system. Grip features can be incorporated molding detents, via spray-on materials, decoration or decoration-like technologies. Non-limiting examples include labels, inks, shrink sleeves and direct printing.

Each plate (2;3) may be independently manufactured from any known material or combination of materials capable of supporting a hair treatment composition. Suitable materials are polymer resins such as a polyolefin e.g. polypropylene, polyethylene or polyethylene terephthalate; biogradable polymers. Other materials which could be used include polyvinylchloride, polyamide, acetyl, acrylonitrile butadiene styrene (ABS), acrylic, acrylonitrile styrene acrylate, ethylene vinyl alcohol, polycarbonate, polystyrene, silicone or thermo plastic elastomer, thermo plastic vulcanate or copolymers where appropriate; flexible pliable substrates such as paper boards, metal based substrates and aluminium foils, filmic substrates or multiple laminations or combinations of multiple layers of said materials. In an embodiment, the present invention may comprise a plate comprising wood, ceramics, glass and metal.

The method of manufacture of the plates (2;3) may include, but is not limited to, injection moulding, co-injection moulding, over moulding, in-mold assembly, compression moulding, blow moulding, thermo or vacuum forming of a blister type shell and lamination onto a carrier plastic or board material in the horizontal or vertical plane. In an embodiment, ribs are created at the plate surface to create an interface for the foam attachment and support. A non-limiting example is ribs can be formed when the molding material is removed to reduce overall resin use and to prevent sink from an overly thick part cross-section.

2. Connection

The selection of a connection (8) to couple together said first (2) and second (3) plates improves the user's perception of control over the hair treatment application device (1) and allows the user to guide the hair treatment application device (1), with the use of either hand, precisely and easily and also allows access to troublesome sections such as the back of the head or the root of the hair close to the scalp. In addition, the connection (8) allows the user to move the hair treatment application device (1) from one hair strand to another without having to adjust the position of the first (2) plate onto the second (3) plate after each application.

The connection (8) between the plates (2;3) according to the present invention allows the hair treatment application device (1) to be in a closed state or in an open state. When the hair treatment application device (1) is in a closed state, said first plate (2) is in juxtaposed relationship to said second plate (3), whereas when the hair treatment application device (1) is in an open state said first plate (2) is distant from said second plate (3). When the hair treatment application device (1) is in an open state, the angle between said internal surfaces (4;5) of said first and second plates (2;3) may range between 5° and 360°, in an embodiment between 30° and 185°, in a further embodiment from about 40° to 75° and in a further embodiment at least 50°. The connection (8) between said first (2) and second (3) plates is may be of resilient nature. In certain embodiments the hair treatment application device (1) may be in an open state and said first (2) and second plates (3) may be brought together into a juxtaposed relationship by applying pressure on their external surfaces (6;7). In certain embodiments the hair treatment application device (1) may be in a closed state and pressure has to be applied on to the connection (8) to distance the internal surfaces (4;5) of the first (2) and the second plate (3). In certain other embodiments, the application device (1) is in a closed state and each plate is provided with fastening means where the thumb and index finger may be positioned to assist the separation of the first and second plates (2,3). Independently from their initial orientation, both the first (2) and the second (3) plates, by pivoting about the connection (8), may re-establish their initial orientation by springing back. The characteristics of the connection (8) may be an intrinsic property of the material used to manufacture the connection or may be provided by the design of the connection itself. The connection (8) should not break or get damaged so as to affect utility within a few applications. On the other end, the connection (8) should not be too loose or else there is no perception of guidance over the hair treatment application device (1). The spring back property should be effective otherwise said first and second plates (2;3) may remain always either in a juxtaposed relationship or distant. In an embodiment of the present invention, the connection (8) may work with applicable pressures ranging from 0.01 N/cm² to 30.0 N/cm², in an embodiment from 0.01 N/cm² to 15.0 N/cm², in a further embodiment from 0.01 N/cm² to 10.0 N/cm² and that the connection should have a spring back pressure to open ranging from 0.01 N/cm² to 30.0 N/cm², in an embodiment from 0.01 N/cm² to 15.0 N/cm² and in a further embodiment from 0.01 N/cm² to 10.0 N/cm².

The first (2) and the second (3) plates are coupled together via any suitable means that fulfils the above described requirements for the connection (8). In one embodiment, each plate (2;3) comprises on the external surface (6;7) a fitting means to accommodate the user's fingers, being the hand of the user the connection.

In a further embodiment, said connection (8) is a hinge. The hinge can be formed in a number of ways including: a “live” injection moulded hinge, a co-injected hinge, an over moulded hinge, in-mold assembly, a leaf spring or any other appropriate spring assembly, a strap hinge, a fold formed by a kiss-cut, score or crease.

In certain embodiments both the first (2) and the second (3) plates have a female part of the hinge incorporated in their design. The female part of the hinge is created during the manufacture process for the first and second plate (2;3), for example during the injection moulding process. A pin is designed to fit both female parts of the hinge created on the first (2) and second plates (3). The pin, in an embodiment of rectangular shape is manufactured from a polymer resin such as polyolefin, in an embodiment, the polymer resign may be polypropylene. The pin is assembled into the female parts of the plates (2;3) to create the hinge.

In an embodiment, the hinge access is at the minor axis of the device and allows more hair to fit in the devise and greater formula surface area exposure for application. The hinge on the minor axis improves the ability to grasp the device and to more efficiently distribute/spread an effective amount of composition on the hair per application.

In an embodiment, the hinge comprises a locking mechanism (9). The locking mechanism (9) may keep the treatment composition or product clean and keeping it from drying out and prevents external contamination. The locking mechanism (9) may be located in a position enabling optimal use and avoiding interaction with the hair during application. In an embodiment, the locking mechanism (9) can be a sliding lock, Velcro, magnets, snapping (with “built in” snap or external snap), hook and eyelet, or an elastic band or other conventional locking mechanism. In an embodiment, the device has a first and second plate (2;3) brought together with a hinge consisting of a sliding lock feature (13) and a hinge spring (12).

In an embodiment, the locking mechanism (9) secures the device in the closed position between uses and for transport/storage. In an embodiment, the locking mechanism (9) is located at the hinge to prevent interaction with the consumers' hair during usage. In an embodiment, the locking mechanism may include a sleeve or a case which serves as a locking mechanism. The locking mechanism is designed to be simple, intuitive and for ease of use.

In an embodiment, the hinge spring (12) enables the device to open (when unlocked) for the user to have optimal grip due to the force generated by the spring. This also provides easier and better access to the formula for hair treatment. The hinge spring (12) is produced in a range of materials, any conventional spring material may be used, and has a torque range between about 0.10in/lbs to about 1.0in/lbs. In a further embodiment, the hinge spring may have applicable pressures ranging from 0.01 N/cm² to 30.0 N/cm², in an embodiment from 0.01 N/cm² to 15.0 N/cm², in a further embodiment from 0.01 N/cm² to 10.0 N/cm² and that the connection should have a spring back pressure to open ranging from 0.01 N/cm² to 30.0 N/cm², in an embodiment from 0.01 N/cm² to 15.0 N/cm² and in a further embodiment from 0.01 N/cm² to 10.0 N/cm².

In certain embodiments, both the first (2) and second (3) plates may be manufactured within the same injection mould for example from polypropylene. A living hinge also made from polypropylene may be created between the first (2) and the second (3) plate. Polypropylene may be used to provide a living hinge that can be flexed multiple times without breakage. The living hinge is typically closed during the de-moulding process.

In certain embodiments, both the first (2) and second (3) plates may be manufactured within the same injection mould for example from polypropylene and a hinge can be created by co-injection, in-mold assembly or over-moulding of a thermo plastic elastomer or a thermo plastic vulcanate or any other material that can be used to provide a hinge with the properties listed above.

In an embodiment of the present invention, the hair treatment application device provides a conductive path from the hair and hand as a means for dissipation of static charge in the hair.

In an embodiment of the present invention, each half of the hinged device performs a function; the bottom half contains the formula and provides a surface for hair to contact, the upper half has a compressed foam surface to press the hair onto the formula surface enabling optimal transfer of formula to hair.

In an embodiment, suitable hair treatment application devices include, but are not limited to, devices composed by a single piece or by a plurality of same or different parts. Examples include, but are not limited to plates attached to handles, tongs, applicators to be fixed to the user's finger, pliable foils, filmic or cellulose based substrates or carrier materials, clips, clamps, shells, pincers, tweezers, scissors, single or multiple moulded parts with flexible elastomers or live hinges, folding combs, not permeable materials, interconnected plates, and in an embodiment hinged plates,

3. Substrate

In an embodiment of the present invention, the device may comprise at least one type of a substrate (10). The substrate may be a deposition aid. In an embodiment, the substrate may be a closed cell compressible foam, a non-woven coated or impregnated with formula, open cell foam (sponge) or like material saturated with formula, flexible wings or other type of flexible material to act as a deposition aid.

In an embodiment, the opposing surface of either the first or second plate containing the hair treatment composition serves as a deposition aid to press the hair to be treated onto the formula surface enabling deposition on the hair. In an embodiment of the present invention, the first and second plate may contain the hair treatment composition. The material of the opposing side and hair treatment composition are produced with a closed cell compressible foam. In a further embodiment, these components could be produced or molded with other conventional materials. A non-limiting example of materials for a closed cell compressible foam are polyethyenes, nylon, polyurethane, resins, polypropylene and other conventional foam material. Anti-static additives and coatings may also be added to the closed cell compressible foam or any of the materials that makeup the substrate. The function of these components are for containing the hair treatment composition, enabling optimal formula deposition (by pressing the hair into the formula), protecting the formula from contamination, enabling consistent transfer of formula to hair throughout usage period, and sealing the clip when not in use for storage/transport (closed). In a further embodiment, a closed cell compressible foam may be used to form a hair treatment composition cavity or well for containing the hair treatment composition.

In an embodiment, the closed cell compressible foam may be affixed to a first or second plate means such as and including two-side tape, thermo-set, hot melt and cold seal, adhesion or extrusion lamination. Mechanical interlock or entanglement such as Velcro®, clamping, snap locks, sealing beads, locking pins and magnetism. In a further embodiment, a substrate can be part of the molding process such as two shot, three shot, multiple shot or sequential molding.

In a further embodiment, suitable absorbent substrates for use in the present invention may be selected from non-wovens; wovens; porous foams and foam materials; porous plastics; flexible frits; meshes; and combinations thereof including recycled and composite materials having one or more plies of the same or different materials superimposed physically, joined together continuously (laminated), in a discontinuous pattern, or by bonding the external edges at discrete loci provided that the structures meet the functional requirements described hereinabove.

In an embodiment, the absorbent substrates of the present invention are preferably selected from non-wovens and/or porous foams.

Non-woven materials are produced from fibers that may be staple or continuous filaments or be formed in situ and include a manufactured sheet, web or batt or directionally or randomly oriented fibers, bonded by friction, and/or cohesion and/or adhesion. Nonwoven webs and processes for making them may comprise three steps: fiber laying, precursor web formation, and fiber bonding. The fiber laying step may be comprised of the spunlaying, meltblowing, carding, airlaying, wetlaying and combinations thereof, of the fibers comprising the web onto a forming surface. The step of precursor web formation may prevent the fibers comprising the web from coming apart during the bonding step. Precursor web formation may be performed via a pre-bonding step, such as one that is chemical or mechanical in nature. The bonding step may then impart strength to the finished web. The bonding step may be comprised of subjecting the fibers comprising the web to hydroentanglement (HET), cold calendering, hot calendering, air through bonding, chemical bonding, needle punching, and combinations thereof. Suitable non-woven materials may be comprised of natural or synthetic fibers selected from acetate fibers; acrylic fibers; cellulose ester fibers; modacrylic fibers; polyamide fibers; polyester fibers; polyolefin fibers; polyvinyl alcohol fibers; rayon fibers; keratin fibers; cellulose fibers; silk fibers and combinations thereof. The non-wovens may be comprised of mono-component fibers, such as a polyolefin or polyester, or bi-component fibers, such as a sheath/core fiber or side by side fiber of polyethylene/polypropylene or polyethylene/polyester, or bi-constituent fibers comprised by a blend of two or more thermoplastic polymers.

In an embodiment, a non-woven substrates may be selected from Carded, Air-laid, and Meltblown non-woven materials or composites. In a further embodiment, the non-woven substrates of the present invention are selected from Carded webs produced by a carding machine with one of more different types of fibres. In a further embodiment, the non-woven substrates of the present invention are selected from multi-layer or lofty web which are consolidated by through air bonding or needle-punching, often referred to as batting battings. Examples of suitable Carded non-wovens for use herein include; Libeltex Thermo-contact 01-766 DI-8; Libeltex Loftfill HC2; PGI FB-215; PGI FB-204B, PGI FB-185 and PGI FB-217.

Porous foams and foam materials are made from low density elastomers, plastics, and other materials with various porosities and may be selected from open cellular foams; flexible foams; rigid foams; and reticular foams and syntactic foams which can be fabricated into finished shapes using molding, casting, extrusion, pultrusion, machining, thermal forming, plastic welding, blow molding, rapid prototyping techniques, grinding and/or other specialized processes. The porous foams and foam materials may be composed of a variety of chemical systems including acrylonitrile-butadiene-styrene (ABS); acrylics; epoxy resins; fluoropolymers; isoprene-styrene (SIS) and styrene-butadiene-styrene (SBS); synthetic rubbers or elastomers based on a variety of systems such as silicone, polyurethane and neoprene; nitrile rubbers; plastics or elastomers formed from natural or plant-based raw materials such as natural rubber (polyisoprene) or vulcanized fibre; water-based and water-borne resins and latex materials. Chemical systems for porous foams and foam materials may include ethylene copolymer, expanded polyethylene, polycarbonate, polyester, polyether, polyetherimide, polyimide, polyamide (nylon), polyolefin, polypropylene, polyurethane, phenolic, polyurea, and vinyl.

Porous plastics can be made from wide variety of materials including Polytetrafluoroethylene (PTFE), Polyethylene (PE), Polypropylene (PP), and Polyvinyldifluoride (PVDF). They are created by filling a mold with tiny plastic pellets, subjecting the mold to heat and pressure so the pellets bond where they touch. This part is then heated outside the mold; the part shrinks significantly during this step which strengthens it.

The porous foams are preferably polyurethane foams. Suitable examples of porous foams are available from Recticel International (Belgium) and include Sweepex S 31 CS/R, Bulpren 528280, Bulpren D32133, Filtren T23220, and Filtren TM 23133.

In a further embodiment, the device of the present invention may be a compact like clip, brush, comb, roller, devise, glove, wipe, stick, or a multi-piece applicator device.

In a further embodiment, the present invention may comprise a non-hinged two piece device that is joined together for storage and transport. The device may be separated (top & bottom halves) for use to enable application of the formulation to the hair. The design to join the halves together may be threaded, snapped, clasped, or some similar method to provide a secure closing feature for the device.

Other Benefit Agents

In one embodiment, the composition may be of any type suitable for application to human hair. In another embodiment, the composition may be of any type suitable for application to pet hair. In yet another embodiment, the composition may be of any type suitable for application to fabric and/or carpet or fibrous substrate.

In one embodiment, the composition may comprise one or more components known for use in hair care or personal care products, provided that the additional components do not otherwise unduly impair product stability, aesthetics, or performance. Such optional ingredients are most typically those described in reference books such as the CTFA Cosmetic Ingredient Handbook, Second Edition, The Cosmetic, Toiletries, and Fragrance Association, Inc. 1988, 1992.

Non-limiting examples of components for use in the composition include conditioning agents (e.g., silicones, hydrocarbon oils, fatty esters), natural cationic deposition polymers, synthetic cationic deposition polymers, anti-dandruff agents, particles, oil absorbing particulate such as tapioca starch, suspending agents, paraffinic hydrocarbons, viscosity modifiers, dyes, non-volatile solvents or diluents (water-soluble and water-insoluble), pearlescent aids, surfactants or nonionic cosurfactants, pediculocides, pH adjusting agents, perfumes, preservatives, proteins, skin active agents, sunscreens, UV absorbers, and vitamins.

1. Conditioning Agent

In one embodiment, the composition may comprise one or more conditioning agents. Conditioning agents include materials that are used to give a particular conditioning benefit to hair and/or scalp. The conditioning agents that may be useful in the composition typically comprise a water-insoluble, water-dispersible, non-volatile, liquid that forms emulsified, liquid particles. Suitable conditioning agents for use in the composition are those conditioning agents characterized generally as silicones (e.g., silicone oils, cationic silicones, silicone gums, high refractive silicones, silicone elastomers and silicone resins), organic conditioning oils (e.g., hydrocarbon oils, polyolefins, and fatty esters) or combinations thereof, or those conditioning agents which otherwise form liquid, dispersed particles in the aqueous surfactant matrix.

One or more conditioning agents may be present from about 1 wt % to about 85 wt %, in an embodiment from about 10 wt % to about 75wt %, and in an embodiment from about 20 wt % to about 0 70 wt %, by weight of the composition.

a. Silicones

The conditioning agent of the composition may be an insoluble silicone conditioning agent. The silicone conditioning agent particles may comprise volatile silicone, non-volatile silicone, or combinations thereof. If volatile silicones are present, it will typically be incidental to their use as a solvent or carrier for commercially available forms of non-volatile silicone materials ingredients, such as silicone gums and resins. The silicone conditioning agent particles may comprise a silicone fluid conditioning agent and may also comprise other ingredients, such as a silicone resin to improve silicone fluid deposition efficiency or enhance glossiness of the hair.

Background material on silicones including sections discussing silicone fluids, gums, and resins, as well as manufacture of silicones, are found in Encyclopedia of Polymer Science and Engineering, vol. 15, 2d ed., pp 204-308, John Wiley & Sons, Inc. (1989), incorporated herein by reference.

i. Silicone Oils

Silicone fluids include silicone oils, which are flowable silicone materials having a viscosity, as measured at 25° C., less than 1000 csk, in an embodiment from about 5 csk to about 200 csk, in a further embodiment 10 to 100 csk. Suitable silicone oils for use in the hair care composition include polyalkyl siloxanes, polyaryl siloxanes, polyalkylaryl siloxanes, polyether siloxane copolymers, and mixtures thereof. Other insoluble, non-volatile silicone fluids having hair conditioning properties may also be used.

Silicone oils include polyalkyl or polyaryl siloxanes which conform to the following Formula (I):

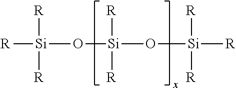

wherein R is aliphatic, in some embodiments alkyl, alkenyl, or aryl, R can be substituted or unsubstituted, and x is an integer from 1 to about 8,000. Suitable R groups for use in the compositions include, but are not limited to: alkoxy, aryloxy, alkaryl, arylalkyl, arylalkenyl, alkamino, and ether-substituted, hydroxyl-substituted, and halogen-substituted aliphatic and aryl groups. Suitable R groups also include cationic amines and quaternary ammonium groups.

Possible alkyl and alkenyl substituents include C₁ to C₅ alkyls and alkenyls, alternativelyfrom C₁ to C₄, and alternatively from C₁ to C₂. The aliphatic portions of other alkyl-, alkenyl-, or alkynyl-containing groups (such as alkoxy, alkaryl, and alkamino) can be straight or branched chains, and may be from C₁ to C₅, alternatively from C₁ to C₄, alternatively from C₁ to C₃, and alternatively from C₁ to C₂. As discussed above, the R substituents can also contain amino functionalities (e.g. alkamino groups), which can be primary, secondary or tertiary amines or quaternary ammonium. These include mono-, di-and tri-alkylamino and alkoxyamino groups, wherein the aliphatic portion chain protrusion length may be as described herein.

ii. Amino and Cationic Silicones

Cationic silicone fluids suitable for use in the composition include, but are not limited to, those which conform to the general formula (II):

(R¹)ₐG₃₋ₐ-Si—(—OSiG₂)ₙ—(—OSiG_{b}(R¹)_{2-b)m}—O—SiG₃₋ₐ(R¹)ₐ

wherein G is hydrogen, phenyl, hydroxy, or C₁-C₈ alkyl, in some embodiments, methyl; a is 0 or an integer having a value from 1 to 3; b is 0 or 1; n is a number from 0 to 1,999, alternatively from 49 to 499; m is an integer from 1 to 2,000, alternatively from 1 to 10; the sum of n and m is a number from 1 to 2,000, alternatively from 50 to 500; R¹ is a monovalent radical conforming to the general formula CqH_{2q}L, wherein q is an integer having a value from 2 to 8 and L is selected from the following groups:

—N(R²)CH₂—CH₂—N(R²)₂

—N(R²)₂

—N(R²)₃A⁻

—N(R²)CH₂—CH₂—NR²H₂A⁻

wherein R² is hydrogen, phenyl, benzyl, or a saturated hydrocarbon radical, in some embodiments an alkyl radical from about C₁ to about C₂₀, and A⁻ is a halide ion.

In one embodiment, the cationic silicone corresponding to formula (II) is the polymer known as “trimethylsilylamodimethicone”, which is shown below in formula (III):

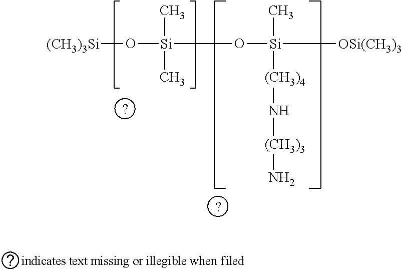

Other silicone cationic polymers which may be used in the composition are represented by the general formula (IV):

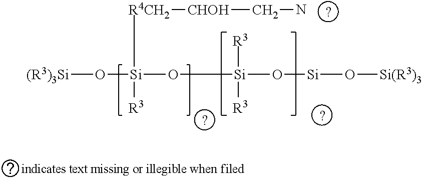

wherein R³ is a monovalent hydrocarbon radical from C₁ to C₁₈, in some embodiments an alkyl or alkenyl radical, such as methyl; R₄ is a hydrocarbon radical, in some embodiments a C₁ to C₁₈ alkylene radical or a C₁₀ to C₁₈ alkyleneoxy radical, alternatively a C₁ to C₈ alkyleneoxy radical; Q⁻ is a halide ion, in some embodiments chloride; r is an average statistical value from 2 to 20, in some embodiments from 2 to 8; s is an average statistical value from 20 to 200, in some embodiments from 20 to 50. One polymer of this class is known as UCARE SILICONE ALE 56®, available from Union Carbide.

iii. Silicone Gums

Other silicone fluids suitable for use in the composition may be insoluble silicone gums. These gums are polyorganosiloxane materials having a viscosity, as measured at 25° C., of greater than or equal to 1,000,000 csk. Silicone gums are described in U.S. Pat. No. 4,152,416; Noll and Walter, Chemistry and Technology of Silicones, New York: Academic Press (1968); and in General Electric Silicone Rubber Product Data Sheets SE 30, SE 33, SE 54 and SE 76, all of which are incorporated herein by reference. Specific non-limiting examples of silicone gums for use in the hair care include polydimethylsiloxane, (polydimethylsiloxane)(methylvinylsiloxane)copolymer, poly(dimethylsiloxane)(diphenyl siloxane)(methylvinylsiloxane)copolymer and mixtures thereof.

iv. High Refractive Index Silicones

Other non-volatile, insoluble silicone fluid conditioning agents that are suitable for use in the hair care composition are those known as “high refractive index silicones,” having a refractive index of at least about 1.46, alternatively at least about 1.48, alternatively at least about 1.52, and alternatively at least about 1.55. The refractive index of the polysiloxane fluid will generally be less than about 1.70, typically less than about 1.60. In this context, polysiloxane “fluid” includes oils as well as gums. The high refractive index polysiloxane fluid includes those represented by general Formula (I) above, as well as cyclic polysiloxanes such as those represented by Formula (V) below:

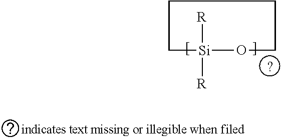

wherein R is as defined above, and n is a number from about 3 to about 7, alternatively from about 3 to about 5.

The high refractive index polysiloxane fluids contain an amount of aryl-containing R substituents sufficient to increase the refractive index to the desired level, which is described herein. Additionally, R and n may be selected so that the material is non-volatile.

Aryl-containing substituents include those which contain alicyclic and heterocyclic five and six member aryl rings and those which contain fused five or six member rings. The aryl rings themselves can be substituted or unsubstituted.

Generally, the high refractive index polysiloxane fluids will have a degree of aryl-containing substituents of at least about 15%, alternatively at least about 20%, alternatively at least about 25%, alternatively at least about 35%, and alternatively at least about 50%. Typically, the degree of aryl substitution will be less than about 90%, more generally less than about 85%, alternatively from about 55% to about 80%. In some embodiments, the high refractive index polysiloxane fluids have a combination of phenyl or phenyl derivative substituents, with alkyl substituents, in some embodiments C₁-C₄ alkyl, hydroxy, or C₁-C₄ alkylamino (especially—R⁴NHR⁵NH2 wherein each R⁴ and R⁵ independently is a C₁-C₃ alkyl, alkenyl, and/or alkoxy).

When high refractive index silicones are used in the composition, they may be used in composition with a spreading agent, such as a silicone resin or a surfactant, to reduce the surface tension by a sufficient amount to enhance spreading and thereby enhance the glossiness (subsequent to drying) of hair treated with the compositions.

Silicone fluids suitable for use in the composition are disclosed in U.S. Pat. No. 2,826,551, U.S. Pat. No. 3,964,500, U.S. Pat. No. 4,364,837, British Pat. No. 849,433, and Silicon Compounds, Petrarch Systems, Inc. (1984), all of which are incorporated herein by reference.

v. Silicone Resins

Silicone resins may be included in composition. These resins are highly cross-linked polymeric siloxane systems. The cross-linking is introduced through the incorporation of trifunctional and tetrafunctional silanes with monofunctional or difunctional, or both, silanes during manufacture of the silicone resin.

Silicone materials and silicone resins in particular, can conveniently be identified according to a shorthand nomenclature system known to those of ordinary skill in the art as “MDTQ” nomenclature. Under this system, the silicone is described according to presence of various siloxane monomer units which make up the silicone. Briefly, the symbol M denotes the monofunctional unit (CH₃)₃SiO_{0.5}; D denotes the difunctional unit (CH₃)₂SiO; T denotes the trifunctional unit (CH₃)SiO_{1.5}; and Q denotes the quadra-or tetra-functional unit SiO₂. Primes of the unit symbols (e.g. M′, D′, T′, and Q′) denote substituents other than methyl, and must be specifically defined for each occurrence.

Silicone resins for use in the composition may include, but are not limited to MQ, MT, MTQ, MDT and MDTQ resins. Methyl is a possible silicone substituent. In some embodiments, silicone resins are MQ resins, wherein the M:Q ratio is from about 0.5:1.0 to about 1.5:1.0 and the average molecular weight of the silicone resin is from about 1000 to about 10,000.

The weight ratio of the non-volatile silicone fluid, having refractive index below 1.46, to the silicone resin component, when used, may be from about 4:1 to about 400:1, alternatively from about 9:1 to about 200:1, and alternatively from about 19:1 to about 100:1, particularly when the silicone fluid component is a polydimethylsiloxane fluid or a mixture of polydimethylsiloxane fluid and polydimethylsiloxane gum as described herein. Insofar as the silicone resin forms a part of the same phase in the compositions hereof as the silicone fluid, i.e. the conditioning active, the sum of the fluid and resin should be included in determining the level of silicone conditioning agent in the composition.

b. Organic Conditioning Oils

The conditioning agent of the composition may also comprise at least one organic conditioning oil, either alone or in combination with other conditioning agents, such as the silicones described above.

i. Hydrocarbon Oils

Suitable organic conditioning oils for use as conditioning agents in the composition may include, but are not limited to, hydrocarbon oils having at least about 10 carbon atoms, such as cyclic hydrocarbons, straight chain aliphatic hydrocarbons (saturated or unsaturated), and branched chain aliphatic hydrocarbons (saturated or unsaturated), including polymers and mixtures thereof. Straight chain hydrocarbon oils may be from about C₁₂ to about C₁₉. Branched chain hydrocarbon oils, including hydrocarbon polymers, typically will contain more than 19 carbon atoms.

ii. Polyolefins

Organic conditioning oils for use in the composition may also include liquid polyolefins, alternatively liquid poly-α-olefins, alternatively hydrogenated liquid poly-α-olefins. Polyolefins for use herein are prepared by polymerization of C₄ to about C₁₄ olefenic monomers, in some embodiments from about C₆ to about C₁₂.

iii. Fatty Esters

Other suitable organic conditioning oils for use as the conditioning agent in the composition may include fatty esters having at least 10 carbon atoms. These fatty esters include esters with hydrocarbyl chains derived from fatty acids or alcohols. The hydrocarbyl radicals of the fatty esters hereof may include or have covalently bonded thereto other compatible functionalities, such as amides and alkoxy moieties (e.g., ethoxy or ether linkages, etc.).

iv. Fluorinated Conditioning Compounds

Fluorinated compounds suitable for delivering conditioning to hair or skin as organic conditioning oils include perfluoropolyethers, perfluorinated olefins, fluorine based specialty polymers that may be in a fluid or elastomer form similar to the silicone fluids previously described, and perfluorinated dimethicones.

v. Fatty Alcohols

Other suitable organic conditioning oils for use in the composition may include, but are not limited to, fatty alcohols having at least about 10 carbon atoms, alternatively from about 10 to about 22 carbon atoms, and alternatively from about 12 to about 16 carbon atoms.

vi. Alkyl Glucosides and Alkyl Glucoside Derivatives

Suitable organic conditioning oils for use in the composition may include, but are not limited to, alkyl glucosides and alkyl glucoside derivatives. Specific non-limiting examples of suitable alkyl glucosides and alkyl glucoside derivatives include Glucam E-10, Glucam E-20, Glucam P-10, and Glucquat 125 commercially available from Amerchol.

c. Other Conditioning Agents

i. Quaternary Ammonium Compounds

Suitable quaternary ammonium compounds for use as conditioning agents in the composition may include, but are not limited to, quaternary ammonium compounds with a long chain substituent having a carbonyl moiety, like an amide moiety, or a phosphate ester moiety or a similar hydrophilic moiety.

Examples of useful quaternary ammonium compounds include, but are not limited to, compounds designated in the CTFA Cosmetic Dictionary as ricinoleamidopropyl trimonium chloride, ricinoleamido trimonium ethylsulfate, hydroxy stearamidopropyl trimoniummethylsulfate and hydroxy stearamidopropyl trimonium chloride, or combinations thereof.

ii. Polyethylene Glycols

Additional compounds useful herein as conditioning agents include polyethylene glycols and polypropylene glycols having a molecular weight of up to about 2,000,000 such as those with CTFA names PEG-200, PEG-400, PEG-600, PEG-1000, PEG-2M, PEG-7M, PEG-14M, PEG-45M and mixtures thereof.

iii. Cationic Polymers

Composition may further comprise a polymer which may be cationic, anionic, nonionic or amphoteric for conditioning benefits or other hair benefits. In an embodiment, such polymers may be present from about 0.01% to about 5%.

d. Anionic Emulsifiers

A variety of anionic emulsifiers can be used in the composition as described below. The anionic emulsifiers include, by way of illustrating and not limitation, water-soluble salts of alkyl sulfates, alkyl ether sulfates, alkyl isothionates, alkyl carboxylates, alkyl sulfosuccinates, alkyl succinamates, alkyl sulfate salts such as sodium dodecyl sulfate, alkyl sarcosinates, alkyl derivatives of protein hydrolyzates, acyl aspartates, alkyl or alkyl ether or alkylaryl ether phosphate esters, sodium dodecyl sulphate, phospholipids or lecithin, or soaps, sodium, potassium or ammonium stearate, oleate or palmitate, alkylarylsulfonic acid salts such as sodium dodecylbenzenesulfonate, sodium dialkylsulfosuccinates, dioctyl sulfosuccinate, sodium dilaurylsulfosuccinate, poly(styrene sulfonate) sodium salt, isobutylene-maleic anhydride copolymer, gum arabic, sodium alginate, carboxymethylcellulose, cellulose sulfate and pectin, poly(styrene sulfonate), isobutylene-maleic anhydride copolymer, gum arabic, carrageenan, sodium alginate, pectic acid, tragacanth gum, almond gum and agar; semi-synthetic polymers such as carboxymethyl cellulose, sulfated cellulose, sulfated methylcellulose, carboxymethyl starch, phosphated starch, lignin sulfonic acid; and synthetic polymers such as maleic anhydride copolymers (including hydrolyzates thereof), polyacrylic acid, polymethacrylic acid, acrylic acid butyl acrylate copolymer or crotonic acid homopolymers and copolymers, vinylbenzenesulfonic acid or 2-acrylamido-2-methylpropanesulfonic acid homopolymers and copolymers, and partial amide or partial ester of such polymers and copolymers, carboxymodified polyvinyl alcohol, sulfonic acid-modified polyvinyl alcohol and phosphoric acid-modified polyvinyl alcohol, phosphated or sulfated tristyrylphenol ethoxylates.

In addition, anionic emulsifiers that have acrylate functionality may also be used in the composition. Anionic emulsifiers useful herein include, but aren't limited to: poly(meth)acrylic acid; copolymers of (meth)acrylic acids and its (meth)acrylates with C1-22 alkyl, C1-C8 alkyl, butyl; copolymers of (meth)acrylic acids and (meth)acrylamide; Carboxyvinylpolymer; acrylate copolymers such as Acrylate/C10-30 alkyl acrylate crosspolymer, Acrylic acid/vinyl ester copolymer/Acrylates/Vinyl Isodecanoate crosspolymer, Acrylates/Palmeth-25 Acrylate copolymer, Acrylate/Steareth-20 Itaconate copolymer, and Acrylate/Celeth-20 Itaconate copolymer; Polystyrene sulphonate, copolymers of methacrylic acid and acrylamidomethylpropane sulfonic acid, and copolymers of acrylic acid and acrylamidomethylpropane sulfonic acid; carboxymethycellulose; carboxy guar; copolymers of ethylene and maleic acid; and acrylate silicone polymer. Neutralizing agents may be included to neutralize the anionic emulsifiers herein. Non-limiting examples of such neutralizing agents include sodium hydroxide, potassium hydroxide, ammonium hydroxide, monoethanolamine, diethanolamine, triethanolamine, diisopropanolamine, aminomethylpropanol, tromethamine, tetrahydroxypropyl ethylenediamine, and mixtures thereof. Commercially available anionic emulsifiers include, for example, Carbomer supplied from Noveon under the tradename Carbopol 981 and Carbopol 980; Acrylates/C10-30 Alkyl Acrylate Crosspolymer having tradenames Pemulen TR-1, Pemulen TR-2, Carbopol 1342, Carbopol 1382, and Carbopol ETD 2020, all available from Noveon; sodium carboxymethylcellulose supplied from Hercules as CMC series; and Acrylate copolymer having a tradename Capigel supplied from Seppic. In another embodiment, anionic emulsifiers are carboxymethylcelluloses.

Other Organic Conditioning Agents

The conditioning agent of the compositions of the present invention may also comprise an additional conditioning agent. The agent can be non-polymeric, oligomeric or polymeric. It can be oil or wax. Examples of conditioning materials include, but are not limited to hydrocarbon oils, polyolefins, fatty esters, metathesized unsaturated polyol esters, sucrose polyesters, fluorinated conditioning compounds, fatty alcohols, alkyl glucosides and alkyl glucoside derivatives, fatty amines, quaternary ammonium compounds; polyethylene glycols, polypropylene glycols, hydrolysed collagen (such as Peptein 2000), vitamin E, panthenol, panthenyl ethyl ether, hydrolysed keratin, proteins, plant extracts and polymers such as chitosan.

Cationic Surfactant

The composition of the invention may contain cationic surfactants. The cationic surfactant may be included in the composition at a concentration of from about 0.2% to about 25%, preferably from about 0.5% to about 15%, more preferably from about 1% to about 10% by weight of the composition. The cationic surfactant may be a mono-alkyl amine cationic surfactant having one long alkyl chain of from 12 to 30 carbon atoms including mono-alkyl amidoamine cationic surfactant. Additionally, di-alkyl quaternized ammonium salt cationic surfactant having two long alkyl chain of from 12 to 30 carbon atoms may be used.

1. Mono-Alkyl Amine Cationic Surfactant

The compositions of the present invention may comprise a mono-alkyl amine cationic surfactant. Mono-alkyl amine cationic surfactants useful herein are primary, secondary, and tertiary amines having one long alkyl or alkenyl group of from about 12 to about 30 carbon atoms, preferably from 16 to 24 carbon atoms, more preferably from 18 to 22 alkyl group. Mono-alkyl amines useful herein also include mono-alkyl amidoamines.

Particularly useful are tertiary amidoamines having an alkyl group of from about 12 to about 22 carbon atoms, preferably from about 16 to about 22 carbon atoms. Exemplary tertiary amido amines include: stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidoprop yldiethyl amine, arachidamidoethyldiethylamine, arachidamidoethyldimethylamine, diethylaminoethylstearamide. Useful amines in the present invention are disclosed in U.S. Pat. No. 4,275,055, Nachtigal, et al.

2.Di-Alkyl Quaternized Ammonium Salt Cationic Surfactant

The composition of the present invention may contain a di-alkyl quaternized ammonium salt cationic surfactant Di-alkyl quaternized ammonium salt cationic surfactants useful herein are those having two long alkyl chains of from 12 to 30 carbon atoms, more preferably from 16 to 24 carbon atoms, still more preferably from 18 to 22 carbon atoms. Such di-alkyl quaternized ammonium salts useful herein are those having the formula (I):

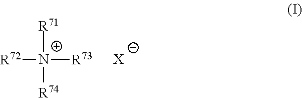

wherein two of R⁷¹, R⁷², R⁷³ and R⁷⁴ are selected from an aliphatic group of from 12 to 30 carbon atoms, preferably from 16 to 24 carbon atoms, more preferably from 18 to 22 carbon atoms or an aromatic, alkoxy, polyoxyalkylene, alkylamido, hydroxyalkyl, aryl or alkylaryl group having up to about 30 carbon atoms; the remainder of R⁷¹, R⁷², R⁷³ and R⁷⁴ are independently selected from an aliphatic group of from 1 to about 8 carbon atoms, preferably from 1 to 3 carbon atoms or an aromatic, alkoxy, polyoxyalkylene, alkylamido, hydroxyalkyl, aryl or alkylaryl group having up to about 8 carbon atoms; and X⁻ is a salt-forming anion selected from the group consisting of halides such as chloride and bromide, C1-C4 alkyl sulfate such as methosulfate and ethosulfate, and mixtures thereof. The aliphatic groups can contain, in addition to carbon and hydrogen atoms, ether linkages, and other groups such as amino groups. The longer chain aliphatic groups, e.g., those of about 16 carbons, or higher, can be saturated or unsaturated. Preferably, two of R⁷¹, R⁷², R⁷³ and R⁷⁴ are selected from an alkyl group of from 12 to 30 carbon atoms, preferably from 16 to 24 carbon atoms, more preferably from 18 to 22 carbon atoms; and the remainder of R⁷¹, R⁷², R⁷³ and R⁷⁴ are independently selected from CH₃, C₂H₅, C₂H₄OH, CH₂C₆H₅, and mixtures thereof.

Such di-alkyl cationic surfactants include, for example, dialkyl (14-18) dimethyl ammonium chloride, ditallow alkyl dimethyl ammonium chloride, dihydrogenated tallow alkyl dimethyl ammonium chloride, distearyl dimethyl ammonium chloride, and dicetyl dimethyl ammonium chloride.

Antistatic Agents

The composition of the current invention may contain an antistatic agent to protect mitigate hair fly-aways. A non-limiting example of an anti-static agent is tricetyl methyl ammonium chloride (TCMAC).

### Benefit Agents

In an embodiment, the composition further comprises one or more additional benefit agents. The benefit agents comprise a material selected from the group consisting of, vitamins, lipid soluble vitamins, chelants, perfumes, brighteners, sensates, attractants, anti-bacterial agents, dyes, pigments, bleaches, hops, caffeine, resorcinol, cleaning agents, and mixtures thereof.

a. Vitamin B₃ Compounds

The composition may include a vitamin B3 compound. In one embodiment, the vitamin B3 compound is niacinamide. Vitamin B3 compounds may be useful for regulating skin conditions, as described in U.S. Pat. No. 5,939,082. In some embodiments, the composition may comprise from about 0.1% to about 25% of a vitamin B3 compound, in another embodiment from about 0.5% to about 15% of a vitamin B3 compound, and in yet another embodiment from about 3.5% to about 7.5% of a vitamin B3 compound. As used herein, “vitamin B3 compound” means a one or more compounds having the formula:

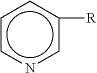

wherein R is —CONH₂ (i.e., niacinamide), —COOH (i.e., nicotinic acid) or —CH2OH (i.e., nicotinyl alcohol); derivatives thereof; mixtures thereof; and salts of any of the foregoing.

Exemplary derivatives of the foregoing vitamin B3 compounds include nicotinic acid esters, including non-vasodilating esters of nicotinic acid (e.g, tocopherol nicotinate, myristyl nicotinate), nicotinyl amino acids, nicotinyl alcohol esters of carboxylic acids, nicotinic acid N-oxide and niacinamide N-oxide. Additional exemplary derivatives of vitamin B3 compounds are set forth in U.S. patent application Ser. No. 11/897,084, which is incorporated herein by reference.

### Fatty Alcohol Gel Network

Embodiments of the composition may also comprise fatty alcohol gel networks, which have been used for years in cosmetic creams and hair conditioners. These gel networks are formed by combining fatty alcohols and surfactants in the ratio of about 1:1 to about 40:1 (alternatively from about 2:1 to about 20:1, and alternatively from about 3:1 to about 10:1). The formation of a gel network involves heating a dispersion of the fatty alcohol in water with the surfactant to a temperature above the melting point of the fatty alcohol. During the mixing process, the fatty alcohol melts, allowing the surfactant to partition into the fatty alcohol droplets. The surfactant brings water along with it into the fatty alcohol. This changes the isotropic fatty alcohol drops into liquid crystalline phase drops. When the mixture is cooled below the chain melt temperature, the liquid crystal phase is converted into a solid crystalline gel network. The gel network contributes a stabilizing benefit to cosmetic creams and hair conditioners. In addition, they deliver conditioned feel benefits for hair conditioners.

Thus according to an embodiment, the fatty alcohol is included in the fatty alcohol gel network at a level by weight of from about 0.05 wt % to about 14 wt %. For example, the fatty alcohol may be present in an amount ranging from about 1 wt % to about 10 wt %, and alternatively from about 6 wt % to about 8 wt %.

The fatty alcohols useful herein are those having from about 10 to about 40 carbon atoms, from about 12 to about 22 carbon atoms, from about 16 to about 22 carbon atoms, or about 16 to about 18 carbon atoms. These fatty alcohols can be straight or branched chain alcohols and can be saturated or unsaturated. Nonlimiting examples of fatty alcohols include, cetyl alcohol, stearyl alcohol, behenyl alcohol, and mixtures thereof. Mixtures of cetyl and stearyl alcohol in a ratio of from about 20:80 to about 80:20, are suitable.

The fatty alcohols useful herein are those having from about 10 to about 40 carbon atoms, from about 12 to about 22 carbon atoms, from about 16 to about 22 carbon atoms, or about 16 to about 18 carbon atoms. These fatty alcohols can be straight or branched chain alcohols and can be saturated or unsaturated. Nonlimiting examples of fatty alcohols include, cetyl alcohol, stearyl alcohol, behenyl alcohol, and mixtures thereof. Mixtures of cetyl and stearyl alcohol in a ratio of from about 20:80 to about 80:20, are suitable.

In one embodiment of the present invention the solid composition contains a particulate material that has the ability to absorb sebum, other oils and residues for hair. The particulate material is deposited upon application on hair, absorbs sebum, oils and other residues and then removed by hair brushing or other means, allowing the consumers to clean and refresh their hair without washing with water. This process saves time and effort not only from avoiding more frequent shampoo washing, but from avoiding frequent styling.

A variety of particulate materials can be used as sebum absorber such as starch or starch derivatives, silicone elastomers, talcum and other inorganic and organic particulate materials. The starch can be corn starch, wheat, potato starch, tapioca starch, rice starch unmodified or chemically modified. Typical examples of chemical modified starch are aluminum octenyl succinate starch and polymethylsilsesquioxane-modified tapioca starch. The later is commercially available with a name of Dry Flo TS.

In an embodiment of the present invention, agents may be added to the hair treatment composition including those for conditioning, smoothing, anti-frizz, fly-away control, anti-static, hair refresher, long lasting scent/fragrance, temporary hair color, styling, damage repair, shine, moisturizing, or cleaning. These benefits might be achieved via adding different actives to the formula, such as UV blockers, oil absorption agents (such as those in dry shampoo), various silicones for smoothing or conditioning, chelants for anti-damage, quats for static, oils/lipids for shine and combinations of these agents.

The composition may optionally contain perfumes to provide deodorizing effect as well as cyclodextrins, which are able to absorb molecules that give malodor, enhancing further the hairrefreshing consumer benefit.

### Encapsulates

The compositions herein may include microcapsules. The microcapsules may be any kind of microcapsule disclosed herein or known in the art. The microcapsules may have a shell and a core material encapsulated by the shell. The core material of the microcapsules may include one or more fragrances. The shells of the microcapsules may be made from synthetic polymeric materials or naturally-occurring polymers. Synthetic polymers can be derived from petroleum oil, for example. Non-limiting examples of synthetic polymers include nylon, polyethylenes, polyamides, polystyrenes, polyisoprenes, polycarbonates, polyesters, polyureas, polyurethanes, polyolefins, polysaccharides, epoxy resins, vinyl polymers, polyacrylates, and mixtures thereof. Non-limiting examples of suitable shell materials include materials selected from the group consisting of reaction products of one or more amines with one or more aldehydes, such as urea cross-linked with formaldehyde or gluteraldehyde, melamine cross-linked with formaldehyde; gelatin-polyphosphate coacervates optionally cross-linked with gluteraldehyde; gelatin-gum Arabic coacervates; cross-linked silicone fluids; polyamine reacted with polyisocyanates; acrylate monomers polymerized via free radical polymerization, and mixtures thereof. Natural polymers occur in nature and can often be extracted from natural materials. Non-limiting examples of naturally occurring polymers are silk, wool, gelatin, cellulose, proteins, and combinations thereof.

The microcapsules may be friable microcapsules. A friable microcapsule is configured to release its core material when its shell is ruptured. The rupture can be caused by forces applied to the shell during mechanical interactions. The microcapsules may have a median volume weighted fracture strength of from about 0.1 MPa to about 25.0 MPa, when measured according to the Fracture Strength Test Method, or any incremental value expressed in 0.1 mega Pascals in this range, or any range formed by any of these values for fracture strength. As an example, the microcapsules may have a median volume weighted fracture strength of 0.5-25.0 mega Pascals (MPa), alternatively from 0.5-20.0 mega Pascals (MPa), 0.5-15.0 mega Pascals (MPa), or alternatively from 0.5-10.0 mega Pascals (MPa).

The microcapsules may have a median volume-weighted particle size of from 2 microns to 80 microns, from 10 microns to 30 microns, or from 10 microns to 20 microns, as determined by the Test Method for Determining Median Volume-Weighted Particle Size of Microcapsules described herein.

The microcapsules may have various core material to shell weight ratios. The microcapsules may have a core material to shell ratio that is greater than or equal to: 10% to 90%, 30% to 70%, 50% to 50%, 60% to 40%, 70% to 30%, 75% to 25%, 80% to 20%, 85% to 15%, 90% to 10%, and 95% to 5%.

The microcapsules may have shells made from any material in any size, shape, and configuration known in the art. Some or all of the shells may include a polyacrylate material, such as a polyacrylate random copolymer. For example, the polyacrylate random copolymer can have a total polyacrylate mass, which includes ingredients selected from the group including: amine content of 0.2-2.0% of total polyacrylate mass; carboxylic acid of 0.6-6.0% of total polyacrylate mass; and a combination of amine content of 0.1-1.0% and carboxylic acid of 0.3-3.0% of total polyacrylate mass.

When a microcapsule's shell includes a polyacrylate material, the polyacrylate material may form 5-100% of the overall mass, or any integer value for percentage in this range, or any range formed by any of these values for percentage, of the shell. As examples, the polyacrylate material may form at least 5%, at least 10%, at least 25%, at least 33%, at least 50%, at least 70%, or at least 90% of the overall mass of the shell.

The microcapsules may have various shell thicknesses. The microcapsules may have a shell with an overall thickness of 1-2000 nanometers, or any integer value for nanometers in this range, or any range formed by any of these values for thickness. As a non-limiting example, the microcapsules may have a shell with an overall thickness of 2-1100 nanometers.

The microcapsules may also encapsulate one or more benefit agents. The benefit agent(s) include, but are not limited to, one or more of chromogens, dyes, antibacterial agents, cooling sensates, warming sensates, fragrances, flavorants, sweeteners, oils, pigments, moldicides, herbicides, phase change materials, adhesives, antioxidants, chelating agents and any other kind of benefit agent known in the art, in any combination. When the benefit agent includes a fragrance, said fragrance may comprise from about 2% to about 80%, from about 20% to about 70%, from about 30% to about 60% of a perfume raw material with a ClogP greater than −0.5, or even from about 0.5 to about 4.5. In some examples, the fragrance encapsulated may have a ClogP of less than 4.5, less than 4, or less than 3. In some examples, the microcapsule may be anionic, cationic, zwitterionic, or have a neutral charge. The benefit agents(s) can be in the form of solids and/or liquids. The benefit agent(s) include any kind of fragrance(s) known in the art, in any combination.

The microcapsules may encapsulate an oil soluble material in addition to the benefit agent. Non-limiting examples of the oil soluble material include mono, di- and tri-esters of C₄-C₂₄ fatty acids and glycerine; isopropryl myristate, soybean oil, hexadecanoic acid, methyl ester, isododecane, and combinations thereof, in addition to the encapsulated benefit agent. The oil soluble material may have a ClogP about 4 or greater, at least 4.5 or greater, at least 5 or greater, at least 7 or greater, or at least 11 or greater.

The microcapsule' s shell may comprise a reaction product of a first mixture in the presence of a second mixture comprising an emulsifier, the first mixture comprising a reaction product of i) an oil soluble or dispersible amine with ii) a multifunctional acrylate or methacrylate monomer or oligomer, an oil soluble acid and an initiator, the emulsifier comprising a water soluble or water dispersible acrylic acid alkyl acid copolymer, an alkali or alkali salt, and optionally a water phase initiator. In some examples, said amine is an aminoalkyl acrylate or aminoalkyl methacrylate.

The microcapsules may include a core material and a shell surrounding the core material, wherein the shell comprises: a plurality of amine monomers selected from the group consisting of aminoalkyl acrylates, alkyl aminoalkyl acrylates, dialkyl aminoalykl acrylates, aminoalkyl methacrylates, alkylamino aminoalkyl methacrylates, dialkyl aminoalykl methacrylates, tertiarybutyl aminethyl methacrylates, diethylaminoethyl methacrylates, dimethylaminoethyl methacrylates, dipropylaminoethyl methacrylates, and mixtures thereof; and a plurality of multifunctional monomers or multifunctional oligomers.

Non-limiting examples of microcapsules include microcapsules that comprise a shell comprising an amine selected from the group consisting of diethylaminoethyl methacrylate, dimethylaminoethyl methacrylate, tertiarybutyl aminoethyl methacrylate; and combinations thereof; a core material encapsulated by said shell, said core material comprising about 10% to about 60% of a material selected from the group consisting of mono, di- and tri-esters of C₄-C₂₄ fatty acids and glycerine; isopropryl myristate, soybean oil, hexadecanoic acid, methyl ester, isododecane, and combinations thereof, by weight of the microcapsule; and about 10% to about 90% of a perfume material, by weight of the microcapsule; wherein said microcapsules have a volume weighted fracture strength from 0.1 MPa to 25 MPa, preferably from 0.8 MPa to 20 MPa, more preferably from 1.0 MPa to 15 MPa; wherein said microcapsules have a median volume-weighted particle size from 10 microns to 30 microns.

Process for making microcapsules are well known. Various processes for microencapsulation, and exemplary methods and materials, are set forth in U.S. Pat. No. 6,592,990; U.S. Pat. No. 2,730,456; U.S. Pat. No. 2,800,457; U.S. Pat. No. 2,800,458; U.S. Pat. No. 4,552,811; and US 2006/0263518 A1.

The microcapsule may be spray-dried to form spray-dried microcapsules. The composition may also contain one or more additional delivery systems for providing one or more benefit agents, in addition to the microcapsules. The additional delivery system(s) may differ in kind from the microcapsules. For example, wherein the microcapsule are friable and encapsulate a fragrance, the additional delivery system may be an additional fragrance delivery system, such as a moisture-triggered fragrance delivery system. Non-limiting examples of moisture-triggered fragrance delivery systems include cyclic oligosaccaride, starch (or other polysaccharide material), starch derivatives, and combinations thereof.

The compositions may also include a parent fragrance and one or more encapsulated fragrances that may or may not differ from the parent fragrance. For example, the composition may include a parent fragrance and a non-parent fragrance. A parent fragrance refers to a fragrance that is dispersed throughout the composition and is typically not encapsulated when added to the composition. Herein, a non-parent fragrance refers to a fragrance that differs from a parent fragrance and is encapsulated with an encapsulating material prior to inclusion into a composition. Non-limiting examples of differences between a fragrance and a non-parent fragrance include differences in chemical make-up.

### I. Solid Compositions

Solid form compositions contain one or more structurants, which may in some instances include one or more waxes, and a surfactant. The solid form compositions are preferably anhydrous. The compositions may also comprise an anhydrous liquid carrier and other components as described below. The compositions are a single phase meaning the final composition is homogenous with respect to active distribution, wax and surfactant concentration. They are not macroscopically segregated by a physical separation into distinct regions of 3D space which could allow for some heterogeneity across the composition. The liquids also do not contain a dispersed liquid phase, microscopically separated such as in an emulsion (water in oil or oil in water). Solid compositions typically have a Durometer A hardness numbers between 3 and 50.

Structurants

The compositions also comprise one or more structurants to provide the solid composition with the desired viscosity, rheology, texture and/or hardness, or to otherwise help suspend any dispersed solids or liquids within the composition. The one or more structurants comprise at least one wax. The term “structurant” may also include any hydrophobic material known or otherwise effective in providing suspending, gelling, viscosifying, solidifying, or thickening properties to the composition or which otherwise provide structure to the solid composition. These structurants may include, for example, gelling agents, polymeric or nonpolymeric agents, inorganic thickening agents, or viscosifying agents. The thickening agents may include, for example, organic solids, silicone solids, crystalline or other gellants, inorganic particulates such as clays or silicas, or combinations thereof.

Waxes may be natural or synthetic materials. In some instances, one or more (or all) of the waxes present in the solid stick antiperspirant composition have a melt temperature less than about 90° C. 85° C., 80° C. 75° C., 70° C. or 60° C. Some examples include natural vegetable waxes such as, for example, candelilla wax, carnauba wax, Japan wax, espartograss wax, cork wax, guaruma wax, rice oil wax, sugar cane wax, ouricury wax, montan wax, sunflower wax, fruit waxes, such as orange waxes, lemon waxes, grapefruit wax, bayberry wax, and animal waxes such as, for example, beeswax, shellac wax, spermaceti, wool wax and uropygial fat. Natural waxes may include the mineral waxes, such as ceresine and ozocerite for example, or the petrochemical waxes, for example petrolatum, paraffin waxes and microwaxes. Chemically modified waxes may be used, such as, for example, montan ester waxes, sasol waxes and hydrogenated jojoba waxes. Synthetic waxes include, for example, a polyethylene, a polymethylene, or a combination thereof.

The wax may also be selected from the group of esters of saturated and/or unsaturated, branched and/or unbranched alkanecarboxylic acids and saturated and/or unsaturated, branched and/or unbranched alcohols, from the group of esters of aromatic carboxylic acids, dicarboxylic acids, tricarboxylic acids and hydroxycarboxylic acids (for example 12-hydroxystearic acid) and saturated and/or unsaturated, branched and/or unbranched alcohols and also from the group of lactides of long-chain hydroxycarboxylic acids. Wax components such as these include, for example, C16-40 alkyl stearates, C20-40 alkyl stearates (for example Kesterwachs (Registered trademark K82H), C20-40 dialkyl esters of dimer acids, C18-38 alkyl hydroxystearoyl stearates or C20-40 alkyl erucates. Other suitable waxes which may be used include C30-50 alkyl beeswax, tristearyl citrate, triisostearyl citrate, stearyl heptanoate, stearyl octanoate, trilauryl citrate, ethylene glycol dipalmitate, ethylene glycol distearate, ethylene glycol di(12-hydroxystearate), stearyl stearate, palmityl stearate, stearyl behenate, cetyl ester, cetearyl behenate and behenyl behenate. Silicone waxes may also be used.

Some preferred examples of acceptable non-polar waxes include glyceryl tribehenate, polyethylene, polymethylene (e.g., Accumelt 68 and 78 available from International Group, Inc., USA), C₁₈-C₃₆ triglyceride (e.g., Synchrowax HGL-C available from Croda, Inc., USA), hydrogenated high euricic aid rapeseed oil (hear stearine), ozokerite and combinations thereof. Some preferred examples of acceptable polar waxes include stearyl alcohol, hydrogenated castor oil, myristyl alcohol, cetyl alcohol, and combinations thereof. The wax may comprise a blend of polar and non-polar waxes. For example, a combination of a polar and non-polar waxes may be selected from the list above. In some instances, the wax may have a melt point above 65° C., more typically from about 65° C. to about 130° C. Some suitable polymethylenes may have a melting point from about 65° C. to about 75° C. Examples of suitable polyethylenes include those with a melting point from about 60° C. to about 95° C. Other high melting point waxes are described in U.S. Pat. No. 4,049,792, Elsnau, issued Sep. 20, 1977. Solid compositions may have a total wax concentration from about 10%, 12%, or 14% to about 25%, 20%, 18% or 16% by weight of the composition.

The compositions may also comprise one or more structurants other than wax. For example, one or more gelling agents may be included. Some non-limiting examples of suitable gelling agents include fatty acid gellants, salts of fatty acids, hydroxyl acids, hydroxyl acid gellants, esters and amides of fatty acid or hydroxyl fatty acid gellants, cholesterolic materials, dibenzylidene alditols, lanolinolic materials, fatty alcohols, triglycerides, sucrose esters such as SEFA behenate, inorganic materials such as clays or silicas, other amide or polyamide gellants, and mixtures thereof.

Suitable gelling agents include fatty acid gellants such as fatty acid and hydroxyl or alpha hydroxyl fatty acids, having from about 10 to about 40 carbon atoms, and ester and amides of such gelling agents. Non-limiting examples of such gelling agents include, but are not limited to, 12-hydroxystearic acid, 12-hydroxylauric acid, 16-hydroxyhexadecanoic acid, behenic acid, eurcic acid, stearic acid, caprylic acid, lauric acid, isostearic acid, and combinations thereof. Preferred gelling agents are 12-hydroxystearic acid, esters of 12-hydroxystearic acid, amides of 12-hydroxystearic acid and combinations thereof.

Other suitable gelling agents include amide gellants such as di-substituted or branched monoamide gellants, monsubstituted or branched diamide gellants, triamide gellants, and combinations thereof, including n-acyl amino acid derivatives such as n-acyl amino acid amides, n-acyl amino acid esters prepared from glutamic acid, lysine, glutamine, aspartic acid, and combinations thereof. Other suitable amide gelling agents are described in U.S. Pat. No. 5,429,816, issued Jul. 4, 1995, and U.S. Pat. No. 5,840,287, filed Dec. 20, 1996.

Still other examples of suitable gelling agents include fatty alcohols having at least about 8 carbon atoms, at least about 12 carbon atoms but no more than about 40 carbon atoms, no more than about 30 carbon atoms, or no more than about 18 carbon atoms. For example, fatty alcohols include but are not limited to cetyl alcohol, myristyl alcohol, stearyl alcohol and combinations thereof.

Non limiting examples of suitable tryiglyceride gellants include tristearin, hydrogenated vegetable oil, trihydroxysterin (Thixcin® R, available from Rheox, Inc.), rape seed oil, castor wax, fish oils, tripalmitin, Syncrowax® HRC and Syncrowax® HGL-C (Syncrowax® available from Croda, Inc.).

Some other structurants for use in the solid compositions may include inorganic particulate thickening agents such as clays and colloidal pyrogenic silica pigments. For example, colloidal pyrogenic silica pigments such as Cab-O-Sil®, a submicroscopic particulated pyrogenic silica may be used. Other known or otherwise effective inorganic particulate thickening agents that are commonly used in the art can also be used in the solid compositions of the present invention. Concentrations of particulate thickening agents may range, for example, from about 0.1%, about 1%, or about 5%; to about 35%, about 15%, about 10% or about 8%, by weight of the composition.

Suitable clay structurants include montmorillonite clays, examples of which include bentonites, hectorites, and colloidal magnesium aluminum silicates. These and other suitable clays may be hydrophobically treated, and when so treated will generally be used in combination with a clay activator. Non-limiting examples of suitable clay activators include propylene carbonate, ethanol, and combinations thereof. When clay activators are present, the amount of clay activator will typically range from about 40%, about 25%, or about 15%; to about 75%, about 60%, or about 50%, by weight of the clay.

A solid composition may contain from about 15% to about 25%, by weight of the composition, of structurants.

Surfactants

The compositions also may comprise one or more surfactants. The surfactant can be any known in the art, including nonionic, cationic, anionic, amphoteric and zwitterionic.

In an embodiment of the present invention, the hair treatment composition is a solid comprising a wax, (low to high molecular weight) silicone, conditioning agent (i.e. amidoamine), a co-solvent, perfume and PMC (perfume micro encapsulate—a solid polyacrylate).

Anhydrous Liquid Carriers

The compositions further comprise one or more anhydrous liquid carriers. These are present, for example, at concentrations ranging from about 10%, about 15%, about 20%, about 25%; to about 99%, about 70%, about 60%, or about 50%, by weight of the composition. Such concentrations will vary depending upon variables such as composition form, desired composition hardness, and selection of other ingredients in the composition. The anhydrous carrier may be any anhydrous carrier known for use in personal care applications or otherwise suitable for topical application to the skin. For example, anhydrous carriers may include, but are not limited to volatile and nonvolatile fluids and combinations thereof.

A. Volatile Fluids

A solid composition may further comprise a volatile fluid, such as a volatile silicone fluid. Volatile fluids are present, for example, at concentrations ranging from about 20% or from about 30%; to about 80%, or no about 60%, by weight of the composition. The volatile silicone of the solvent may be cyclic, linear, and/or branched chain silicone. “Volatile silicone”, as used herein, refers to those silicone materials that have measurable vapor pressure under ambient conditions. Non-limiting examples of suitable volatile silicones are described in Todd et al., “Volatile Silicone Fluids for Cosmetics”, Cosmetics and Toiletries, 91:27-32 (1976).

The volatile silicone may be a cyclic silicone. The cyclic silicone may have from about 3 silicone atoms, or from about 5 silicone atoms; to about 7 silicone atoms, or about 6 silicone atoms. For example, volatile silicones may be used which conform to the formula:

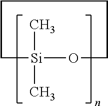

wherein n is from about 3, or from about 5; to about 7, or about 6. These volatile cyclic silicones generally have a viscosity of less than about 10 centistokes at 25° C. Suitable volatile silicones for use herein include, but are not limited to, Cyclomethicone D5 (commercially available from G. E. Silicones); Dow Corning 344, and Dow Corning 345 (commercially available from Dow Corning Corp.); and GE 7207, GE 7158 and Silicone Fluids SF-1202 and SF-1173 (available from General Electric Co.). SWS-03314, SWS-03400, F-222, F-223, F-250, F-251 (available from SWS Silicones Corp.); Volatile Silicones 7158, 7207, 7349 (available from Union Carbide); Masil SF-V (available from Mazer) and combinations thereof.

B. Non-Volatile Fluids

The non-volatile organic fluid can be present, for example, at concentrations ranging from about 1% to about 90% by weight of the composition.

1. Non-Volatile Organic Fluids

The non-volatile organic fluid can be present at concentrations ranging from about 1%, from about 2% but no more than about 20% or no more than about 15%, by weight of the composition.

Non-limiting examples of nonvolatile organic fluids include, but are not limited to, mineral oil, PPG-14 butyl ether, isopropyl myristate, petrolatum, butyl stearate, cetyl octanoate, butyl myristate, myristyl myristate, C12-15 alkylbenzoate (e.g., Finsolv™), dipropylene glycol dibenzoate, PPG-15 stearyl ether benzoate and blends thereof (e.g. Finsolv TPP), neopentyl glycol diheptanoate (e.g. Lexfeel 7 supplied by Inolex), octyldodecanol, isostearyl isostearate, octododecyl benzoate, isostearyl lactate, isostearyl palmitate, isononyl/isononoate, isoeicosane, octyldodecyl neopentanate, hydrogenated polyisobutane, and isobutyl stearate. Many such other carrier liquids are disclosed in U.S. Pat. No. 6,013,248 (Luebbe et al.) and U.S. Pat. No. 5,968,489 (Swaile et al).

2. Non-Volatile Silicone Fluids

The composition may further comprise a non-volatile silicone fluid. The non-volatile silicone fluid may be a liquid at or below human skin temperature, or otherwise in liquid form within the anhydrous composition during or shortly after topical application. The concentration of the non-volatile silicone may be from about 1% to about 90% by weight of the composition. Non-volatile silicone fluids may include those which conform to the formula:

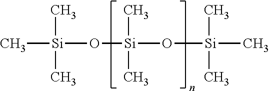

wherein n is greater than or equal to 1. These linear silicone materials may generally have viscosity values of from about 5 centistokes, from about 10 centistokes; to about 100,000 centistokes, about 500 centistokes, about 200 centistokes, or about 50 centistokes, as measured under ambient conditions.

Specific non limiting examples of suitable nonvolatile silicone fluids include Dow Corning 200, hexamethyldisiloxane, Dow Corning 225, Dow Corning 1732, Dow Corning 5732, Dow Corning 5750 (available from Dow Corning Corp.); and SF-96, SF-1066 and SF18(350) Silicone Fluids (available from G.E. Silicones).

Low surface tension non-volatile solvent may be also be used. Such solvents may be selected from the group consisting of dimethicones, dimethicone copolyols, phenyl trimethicones, alkyl dimethicones, alkyl methicones, and mixtures thereof. Low surface tension non-volatile solvents are also described in U.S. Pat. No. 6,835,373 (Kolodzik et al.).

Methods

In an embodiment of the present invention, the hair treatment composition may be made as follows: When the formula contains stearamidopropyl dimethylamine SAPDMA a premix is made wherein the SAPDMA and cosolvent* are mixed together at ambient conditions. SAPDMA concentration should be between 20-30% of the premix composition. The premix is heated with mixing to +/−5° C. of the main mix final temperature. *Cosolvents are phenyl trimethicone or Finsolv TN

The main mix is then prepared wherein addition of all other ingredients together in the main mix is completed. The main mix is heated to 15-25° C. above the highest melt point material used while mixing. The premix is then added to the main mix at temperature, while mixing. And proceeded to be mixed for 5 minutes minimum. The heat is turned off to the batch, allowing to slowly cool while mixing. Batch is then poured into device when temperature is 5-15° C. above the highest melt point material used.

For formulae without stearamidopropyl dimethylamine (SAPDMA):

All of the ingredients are added together in the main mix. The main mix is then heated to 15-25° C. above the highest melt point material used while mixing. The mix is then mixed a minimum of 5 minutes at temperature. Turn off heat to batch, and allow to slowly cool while mixing. Pour batch into device when temperature is 5-15° C. above the highest melt point material used.

Melt Point Method—In an embodiment of the present invention, the melt point method varies based on type of wax used in the hair treatment composition. Process: Weigh materials into vessel, heat and agitate materials to melt point, increase temperature 20-30 C above melt point, add any remaining materials or pre-mixes to complete formula, hold at temperature until all materials incorporated (uniform/single phase formula), reduce temperature, pour formula into device at 5-15 C above the highest melt point material used.

In an embodiment of the present invention, the hardness of the hair treatment composition may be measured using any conventional method. In an embodiment of the present invention the hardness of the hair treatment method may be measured as follows: Durometer A Hardness Measurement: Equilibrate sample to be measured at 22 C±2 C at 36% rh±5%, place sample on Durometer measurement platform, zero gauge, initiate measurement by lowering Durometer gauge probe to contact sample surface, time 5 seconds from point of probe/sample contact, record gauge number at 5 seconds (retention number) and maximum number (number generated at initial probe & sample contact). In an embodiment, a Durometer OO may be used following the procedure above.

In an embodiment of the present invention, hair treatment compositions with a Durometer A hardness numbers <3 result in a formula surface that is too soft and does not provide an optimal usage experience, i.e. greasy appearance, white residue on hair. In an embodiment of the present invention, hair treatment composition with a Durometer A hardness number >50 results in a formula with a plastic like surface/material and little to no transfer of formula to the hair.

In an embodiment of the present invention, the present invention may have a hair treatment composition with a Durometer A hardness in a range of 3-50 (retention at 5 sec.), in an embodiment a Durometer A hardness of 5-40 (retention at 5 sec.), in a further embodiment, a Durometer A hardness of 10-20 (retention at 5 sec.).

Melt Point: This number is generated based on materials used in formula and visual observations. As the formulation is heated and agitated the mix becomes a liquid when the solids (waxes) have melted—this is the measured melt point.

Deposition Method: Product is applied via delivery system onto 4 g/6″ general population, medium brown hair tress. Samples to be measured are equilibrated at 22 C±2 C at 36% rh±5% (hair tress and filled delivery system). Hair tress weighed prior to application and weight recorded, tress hung in vertical orientation, filled delivery system, ‘swiped’ from top to end of tress 10 times, treated tress weighed and weight recorded. Visual observations of hair tress condition recorded.

Deposition—Hair Benefit:

In an embodiment of the present invention, application of hair treatment composition (10 strokes) from top to bottom on a hair tress may result in a deposition of 0.03 to 0.18 grams of material to hair tress resulting in positive benefits without negative hair appearance. In a further embodiment, application of hair treatment composition (10 strokes) from top to bottom on a hair tress may result in a deposition of 0.05 to 0.15 grams of material to hair tress resulting in positive benefits without negative hair appearance.

TABLE 1
Impact of Formula Hardness
Product Description: Frizz Buster Formula
Process: Traditional Lab

| | Comparative | Comparative | Invention | Invention | Invention | Invention |
|---|---|---|---|---|---|---|
| | Example 1t) | Example 2 | Example 3 | Example 4 | Example 5 | Example 6 |
| | Weight % | Weight % | Weight % | Weight % | Weight % | Weight % |
| Raw Materials | | | | | | |
| Cyclomethicone¹ | 28.80 | | | | | |
| 10 cst Dimethicone² | 50.00 | 85.00 | 73.00 | 71.00 | 68.50 | 63.00 |
| Stearyl Alcohol³ | 12.00 | | | | | |
| Performalene 1290⁴ | | 12.00 | 25.00 | 20.00 | 22.50 | 25.00 |
| R5426A⁵ | | | | | | |
| White Ozokerite Wax⁶ | 3.00 | | | | | |
| Behenyl Alcohol⁷ | 0.20 | | | | | |
| Mineral Oil⁸ | 5.00 | | | | | |
| PMC Apple Bloom (90/10)⁹ | | 0.75 | | 1.00 | 1.00 | 1.00 |
| Quaternium-80¹⁰ | | 1.25 | | | | |
| Phenyl Trimethicone¹¹ | | | | 5.00 | 5.00 | 8.00 |
| SAPDMA¹² | | | 2.00 | 2.00 | 2.00 | 2.00 |
| (stearamidopropyl- | | | | | | |
| dimethylamine) | | | | | | |
| C12-15 Alkyl | | | | | | |
| Benzoate¹³ | | | | | | |
| Dimethicone & Dimethiconol¹⁴ | | | | | | |
| Fragrance | 1.00 | 1.00 | | 1.00 | 1.00 | 1.00 |
| Total | 100.00 | 100.00 | 100.00 | 100.00 | 100.00 | 100.00 |
| Hardness (Durometer Gauge A) | | | | | | |
| Retention # at 5 seconds | Too soft | 1 | 3 | 7 | 7 | 10 |
| Maximum | | 5 | 9 | 22 | 21 | 25 |
| Hair Application Observations | Greasy, | Greasy, | Good | Good | Good | Good |
| (10 swipes on | White | White | Appearance | Appearance | Appearance | Appearance |
| 4 g/6″ hair tress) | Residue | Residue | | | | |
| Formula Deposition on Tress | 0.1 g | 0.19 g | 0.0 | 0.09 g | 0.03 g | 0.1 g |
| (10 swipes on | | | | | | |
| 4 g/6″ hair tress) | | | | | | |

| | |
|---|---|
| Unacceptable hair tress appearance | Acceptable hair tress appearance |

| | Invention | Invention | Invention | Comparative | Comparative |
|---|---|---|---|---|---|
| | Example 7 | Example 8 | Example 9 | Example 10 | Example 11 |
| | Weight % | Weight % | Weight % | Weight % | Weight % |
| Raw Materials | | | | | |
| Cyclomethicone¹ | | | | | |
| 10 cst Dimethicone² | 63.00 | 53.00 | 45.00 | 12.00 | 12.00 |
| Stearyl Alcohol³ | | | | | |
| Performalene 1290⁴ | 25.00 | | | 85.00 | 84.25 |
| R5426A⁵ | | 35.00 | 37.00 | | |
| White Ozokerite Wax⁶ | | | | | |
| Behenyl Alcohol⁷ | | | | | |
| Mineral Oil⁸ | | | | | |
| PMC Apple Bloom (90/10)⁹ | 1.00 | 1.00 | 1.00 | 0.75 | 0.75 |
| Quaternium-80¹⁰ | | | | 1.25 | |
| Phenyl Trimethicone¹¹ | | | | | |
| SAPDMA¹² | 2.00 | 2.00 | 8.00 | | |
| (stearamidopropyl- | | | | | |
| dimethylamine) | | | | | |
| C12-15 Alkyl | 8.00 | 8.00 | 8.00 | | |
| Benzoate¹³ | | | | | |
| Dimethicone & Dimethiconol¹⁴ | | | | | 2.00 |
| Fragrance | 1.00 | 1.00 | 1.00 | 1.00 | 1.00 |
| Total | 100.00 | 100.00 | 100.00 | 100.00 | 100.00 |
| Hardness (Durometer Gauge A) | | | | | |
| Retention # at 5 seconds | 14 | | 7 | 51 | 54 |
| Maximum | 26 | | 15 | 68 | 76 |
| Hair Application Observations | Good | Good | Good | No | No |
| (10 swipes on | Appearance | Appearance | Appearance | transfer | transfer |
| 4 g/6″ hair tress) | | | | | |
| Formula Deposition on Tress | 0.07 g | 0.1 g | 0.11 g | 0.00 | 0.00 |
| (10 swipes on | | | | | |
| 4 g/6″ hair tress) | | | | | |

| | |
|---|---|
| Acceptable hair tress appearance | No benefit delivered to hair tress |

Raw Materials
¹Cyclopentasiloxane, Dow Corning 245 Fluid, Dow Corning
²Dimethicone, Dow Corning 200 fluid, 10 cst, Dow Corning Corp
³Stearyl Alcohol NF, Stearyl Alcohol NF, P&G Manufacturing
⁴Polyethylene^{,} Performalene 1290, Baker Petrolite Polymers
⁵Polymethylene, R5426A, International Waxes, Inc.
⁶ White Ozokerite Wax, White Ozokerite Wax Pastilles SP-1026P, Strahl & Pitsch Inc
⁷Behenyl Alcohol, Behenyl Alcohol, BASF Corp
⁸Mineral Oil, Carnation White Mineral Oil, Sonneborn Inc
⁹ Perfume Microcapsule PMC Apple Bloom 52C, Spray Tek Inc
¹⁰ Quaternium-80, ABIL ® Quat 3272, Evonik Industries AG
¹¹Phenyl Trimethicone, Dow Corning 556 Cosmetic Grade Fluid, Dow Corning Corp
¹²Stearamidopropyl Dimethylamine, Incromine SBPG/Incromine SD-(PG)-PA-(MH), Croda Inc.
¹³C12-15 Alkyl Benzoate, Finsolv TN from Innospec Active Chemicals LLC
¹⁴Dimethicone and Dimethiconol (Polydimethylsiloxane and dimethyl siloxane hydroxy terminated), Dow Corning 1503 Fluid, Dow Corning
Fragrance

Table 1 provides Examples of the present invention (Examples 3-8) and comparative examples (Examples 1, 2, 9 and 10). The examples are evaluated for deposition weight, qualitative assessment of hair tress and hardness measurement. The Deposition and Assessment Method for examples in Table 1 is Hair Tress (4 gram/6″ tress) are weighed before formula application. Application of formula (10 strokes) is applied room top to bottom on a hair tress. The treated hair tress is then weighed and weight recorded. A visual assessment of hair tress for positive and negative appearance is performed (i.e. acceptable, not acceptable, greasy, residue) and observations are recorded

The results of the table provide that Examples 3-8 provide the desired benefits for hardness, deposition and appearance. Examples 3-8 are representative of the present invention. Example land 2 and Example 9 and 10 are comparative examples. Examples 1 and 2 would not deliver the desired benefit that the present invention delivers because they delivered unacceptable hair tress appearance. Examples 9 and 10 would not deliver the desired benefit that the present invention delivers because these examples did not deliver the hair treatment composition to the hair tress.

Table 2 provides Consumer-related evaluations data (or technical evaluation data that correlated with specific consumer benefits) from a comparative example 11 and a commercial anti-frizz liquid product on a non-woven substrate. The evaluation is a test with 20 people for one week time and evaluates wipes with commercial anti-frizz formula which is a liquid vs. a solid treatment composition. Values in table 2 are based on a 5 point scale with values 0-100. This study further provides supporting data showing that comparative example 1 does not provide the desired benefits of appearance, due to lack of desired hardness and high deposition of materials results in a negative appearance versus a conventional anti-frizz formula which is a liquid applied to non-woven substrates.

TABLE 2

| | | Example 12 Commercial |
|---|---|---|
| | Comparative | Anti-Frizz formula (liquid) |
| LOT Code | Example 1 | on a Non-woven Substrate |
| Not leaving hair greasy | 32 | 60 S |
| Not leaving residue on Hair | 32 | 59 S |
| Not weighing hair down | 33 | 57 S |

Qualitative Static Results: Static Results as SAPDMA Level and Hair Damage Vary

TABLE 3

| SAPDMA | Hair Damage: | |
|---|---|---|
| (Stearamidopropyl | 0 = Virgin | Qualitative Static Value: |
| Dimethylamine Level) | 2 = Low Lift 2 | 0 = No Static |
| % | (damaged) | 4 = Excessive Static |
| 2 | 0 | 3 |
| 2 | 2 | 3 |
| 5 | 0 | 0 |
| 5 | 2 | 1 |
| 8 | 0 | 0 |
| 8 | 2 | 0 |

Methodology: Table 3—Three swipes with formula containing hair treatment application delivery system using a polyethylene foam is performed on hair switches. All testing is performed in a controlled temperature and humidity room with a dew point of 9.5° Celsius. The switches are 8″ long (including binding) and contain 4 g of hair. 0=no static fly, 1=hair switch is slightly puffy or has a few strands slightly out of place; 3=more strands out of place and 4=excessive number of strands out of place.

Results: The Table 3 indicates that static fly is generated at 2% SAPDMA for both hair types. Increasing SAPDMA level to 5% indicates only slight static generated on damaged hair. Additional SAPDMA increase to 8% eliminates static fly for both virgin and damaged hair.

In the examples, all concentrations are listed as weight percent, unless otherwise specified and may exclude minor materials such as diluents, filler, and so forth. The listed formulations, therefore, comprise the listed components and any minor materials associated with such components. As is apparent to one of ordinary skill in the art, the selection of these minors will vary depending on the physical and chemical characteristics of the particular ingredients selected to make the hair care composition.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hair treatment application delivery system comprising:
a) at least one solid treatment composition comprising a wax and a silicone;
b) a hair treatment application delivery device comprising a first plate and a second plate positionable in a juxtaposed relationship when the hair treatment application delivery device is in a closed state, wherein the first and second plates are coupled together via a connection, and wherein the first and second plates each has an internal and external surface.

2. The hair treatment application delivery system according to claim 1 wherein at the first plate comprises a substrate.

3. The hair treatment application delivery system according to claim 2 wherein the first plate comprises a closed cell compressible foam.

4. A composition according to claim 1 wherein the treatment composition further comprises a cationic surfactant.

5. A composition according to claim 4 wherein the cationic surfactant is an amine or a quaternary ammonium salt.

6. A composition according to claim 5 wherein the cationic surfactant is stearamidopropyl dimethylamine.

7. A hair treatment application delivery system according to claim 1 wherein the treatment composition is free of volatile solvents or carriers.

8. A hair treatment application delivery system according to claim 1 wherein the treatment composition has a hardness in a range of 3-50 retention of 5 seconds.

9. A hair treatment application delivery system according to claim 1 wherein the treatment composition is deposited on hair at a deposition of 0.03 to 0.18 grams.

10. A hair treatment application delivery system according to claim 1 wherein the connection comprises a spring back pressure to open ranging from 0.01N/cm² to 20 N/cm².

11. A hair treatment application delivery system according to claim 1 wherein the connection is a hinge.

12. A hair treatment application delivery system according to claim 1 wherein the connection further comprises a spring.

13. A hair treatment application delivery system according to claim 1 wherein the connection comprises a locking mechanism.

14. A hair treatment application delivery system according to claim 1 wherein the external surface of the first and/or the second plate further comprises visible features or tactile materials.

15. A hair treatment application delivery system according to claim 1 wherein the hair treatment application delivery system comprises a clip, brush, comb, roller, glove, wipe, stick, or a multi-piece applicator device.

16. A method of treating frizz and flyaways on hair comprising the step of using the hair treatment application delivery system of claim 1.
